(19) United States
(12) Patent Application Publication
(10) Pub. No.: US 2001/0043951 A1
(43) Pub. Date: Nov. 22, 2001

(54) Polyphosphate for use in promoting wound healing and scar abatement

(75) Inventors: Hong Yeoul Kim, Seoul (KR); Ho Yeon Lee, Seoul (KR)

Correspondence Address: GATES & COOPER, Howard Hughes Center, Suite 1050, 6701 Center Drive West, Los Angeles, CA, 90045, US

(73) Assignee: KYUNG WON MEDICAL CO., LTD.

(21) Appl. No.: 09/818,007

(22) Filed: Mar. 26, 2001

(30) Foreign Application Priority Data
Mar. 30, 2000 (KR) KR2000-16691

(51) Int. Cl. A61K 33/42; C01B 25/12

(52) U.S. Cl. 424/601; 424/606; 423/304

(57) ABSTRACT

Disclosed is a polyphosphate for use in promoting wound healing and scar abatement, represented by the following chemical formula 1. The polyphosphate can effectively promote the healing of wounds resulting from surgical operation or cutting without leaving scars, thereby encouraging patients to overcome loss of self-esteem. In addition, the medicinal aid can heal wounds effectively and economically while lessening scars.

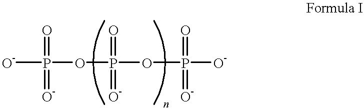

wherein n is an integer from 3 to 798.

This application claims benefit under 35 U.S.C. §119 to Korean patent application number 2000-16691, filed Mar. 30, 2000, the entire contents of which are incorporated herein by reference.

### BACKGROUND OF THE INVENTION

1. Field of the invention

The present invention relates to use of polyphosphate in promoting wound healing and scar abatement. More particularly, the present invention relates to polyphosphate which, upon being applied to wounds such as cuts or surgical operation sequelae, can effectively heal the wounds with leaving no or almost no scars, thereby aiding the recovery of patients and encouraging patients to overcome loss of self-esteem which may result from the formation of scars.

2. Description of the Prior Art

Polyphosphate is a linear polymer of many tens or hundreds of orthophosphate (Pi) residues linked by high-energy, phosphoanhydride bonds. In vivo, ends of inorganic polyphosphate chains are attacked by AMP, ADP, glucose and H₂O during the catalytic action of P-AMP phosphotransferase, polyphosphate kinase, polyphosphate glucokinase and exopolyphosphatase, respectively. Endogenously, inorganic polyphosphate is hydrolyzed by endopolyphosphatase.

Polyphosphate is found in a broad spectrum of living cells. Scientists believe that in animals, one of its roles may be to serve as a phosphate storage reservoir for the production of ATP (adenosine triphosphate), which provides the energy to power a cell. Recently, it has been disclosed that in bacteria, polyphosphate helps these single-celled organisms adapt to nutritional deficiencies and environmental stresses. For example, when bacteria are subjected to nutritional deficiencies or environmental stresses (e.g., heat or osmotic pressure), polyphosphate is synthesized to supply the energy necessary for the production of various proteins.

In addition, polyphosphate has been suggested to have antibacterial activity, but the suggestion has not been demonstrably proven through experiments (Brown. A. T and R. RUH, JR. 1977. Negative interaction of orthophosphate with glycolytic metabolism by Streptococcus mutants as a possible mechanism for dental caries reduction. Archs Oral. 22:521-524; Knabel. S. J., H. W. Walker and P. A. Hrtman. 1991. Inhibition of Aspergillus flavus and selected Gram-positive bacteria by chelation of essential metal cations by polyphosphate. 1991. KIM and LEE KR99-6697, Journal of Food Protection. 50:360-365; Tanzer J. M. and G. J. Hageage, JR., 1970, Polyphosphate inhibition of growth of plaques formed by Streptococci and Diphterides implicated in oral disease. Infection and Immunity 604-606; and Laula L. Zaika and Anna H. Kim, 1993, Effect of sodium polyphosphate on growth of Listeria monocytogens. Journal of Food Protection 56:577-580).

Being highly negatively charged, polyphosphate can strongly associate with polyvalent cations, such as cobalt ions, copper ions, magnesium ions, calcium ions, iron ions, zinc ions, etc. For this reason, that is, because polyphosphate can stably combine with the materials microorganisms take up through normal metabolism, the inorganic polymer is inhibitory against the growth of microorganisms.

A variety of phosphates have long been used to protect foods from moisture; to restrain the formation of suspension in foods; to prevent foods from being shrunk upon cooking; and to soften foods, such as meats. In addition, polyphosphates find applications in various fields as color developers for meats and cheeses, removers of curds from canned foods, additives for carbonated water, dentifrices and tums and have gained an approval from the FDA and Department of Agriculture of U. S. A.

In spite of its ubiquitousness in every living cell, polyphosphate has not yet been fully understood as to its functions. In fact, polyphosphate, although being extensively utilized in a wide range of fields as described above, has been studied for its inhibitory activity against some bacteria, but has received little attention in other fields.

As a result of some research with Gram-positive bacteria, polyphosphate is found to be inhibitory against oral bacteria (Brown. A. T and R. RUH, JR. 1977. Negative interaction of orthophosphate with glycolytic metabolism by Streptococcus mutants as a possible mechanism for dental caries reduction. Archs Oral. 22:521-524; Knabel. S. J., H. W. Walker and P. A. Hrtman. 1991. KIM and LEE KR 2000-16691 Inhibition of Aspergillus flavus and selected Gram-positive bacteria by chelation of essential metal cations by polyphosphate. 1991. Journal of Food Protection. 50:360-365; and Tanzer J. M. and G. J. Hageage, JR., 1970, Polyphosphate inhibition of growth of plaques formed by Streptococci and Diphterids implicated in oral disease. Infection and Immunity 604-606). Some linear sodium polyphosphates, such as disodium phosphate, sodium pyrophosphate, tetrasodium pyrophosphate, sodium tripolyphosphate, sodium tetrametaphosphate, and sodium hexametaphosphate, were restrictively examined for antibacterial activity against Staphylococcus aureus (Chung Mei C. Jen and Leora A. Shelf. 1986, Factors affecting sensitivity of Staphylococcus aureus 196E to Polyphosphates. Applied and Environmental Microbiology, 842-846) or Streptococcus mutant. There has been reported no research on the use of polyphosphate in recovering wounds and in suppressing scar formation, thus far.

Polyphosphate, especially, a chain of around 75 orthophosphates, is known to be not only used where osteogenesis is needed, but also useful for osteoregeneration when bones are removed as a result of a surgical operation or when bones are required to be substituted or regenerated for other reasons. Another application of polyphosphate is found where bone grafting or prosthesis is conducted. Polyphosphate helps the bone onlay or the prosthesis quickly adapt to the new environment, thereby promoting osteogenesis. However, regarding the promotion of wound healing and scar restraint, no effects have been discovered using polyphosphate.

When wounds are generated on the skin as a result of surgical operation, cutting, scratching, sting, abscess, dermatitis, etc., they are generally treated by coating medicines of germicides and astringents in combination. Typically, these topical medicines exert their therapeutic effects by forming a mucous membrane or a thin film on the wound to protect it from being infected, contracting blood vessels around the wound, restraining the secretion of humor to the wound to keep it dry, and killing germs around the wound.

Conventional dermal medicines for topical use are, however, so quickly dried as to contract the skin surrounding the wound, resulting in leaving scars after wounds have healed. Also, conventional dermal medicines suffer from drawbacks of requiring a long period of time for wound healing because the mucous membranes or thin films formed on wounds are so poor in selective permeability as for air and therapeutically effective ingredients not to penetrate effectively through the membranes or films.

When a deep wound is generated in a large area on the skin, it must be scrupulously sewed up in order not to leave a serious scar because it does not vanish with the use of conventional topical ointments. Accordingly, not only does it take a long time period to suture the wound, but also intensive attention must be paid to the suturing. Hyperbaric oxygen therapy is useful in treating deep wounds without leaving scars, but is complicated and costs a great deal.

### SUMMARY OF THE INVENTION

Leading to the present invention, intensive research on wound healing, conducted by the present inventors, resulted in the finding that polyphosphate can promote the healing of wounds caused by surgical operation, cutting, injury, abscess, dermatitis, etc., with suppression of scaring.

Therefore, it is an object of the present invention to provide the use of polyphosphate in healing wounds, such as surgical operation sequelae, cuts, scratches, abscesses, dermatitis, etc., without leaving scars, thereby encouraging patients to overcome loss of self-esteem.

In accordance with the present invention, there is provided a medicinal aid for use in promoting wound healing and scar abatement, comprising as therapeutically effective ingredient a polyphosphate represented by the following chemical formula 1:

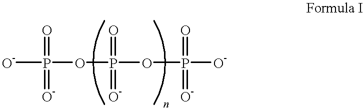

wherein n is an integer from 3 to 798.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 is a graph showing the progress of wound healing with time, in accordance with Example 1.

FIG. 2 shows the therapeutic effect of the medicinal aid of Example 2 in photographs of wounds taken at 3 days (a), 6 days (b), 10 days (c), 14 days (d), and 15 days (e) after the operation.

FIGS. 3 to 6 are photographs showing healing progresses of wounds to which medicinal aids of Examples 3 to 6 are applied.

FIG. 7 shows the therapeutic effect of the medicinal aid of Comparative Example 1 in photographs of wounds taken at 3 days (a), 6 days (b), 10 days (c), 14 days (d), and 15 days (e) after the operation.

FIG. 8 shows photographs illustrating the result obtained in Comparative Example 2.

### DETAILED DESCRIPTION OF THE INVENTION

Useful for promoting wound healing and scar declination in accordance with the present invention are straight-chain polyphosphate obtained through the dehydropolycondensation of ortho-phosphates, branched polyphosphates grafted with organic groups, and cyclic polyphosphates. Of them, straight-chain polyphosphates represented by the following chemical formula 1, in which two or more PO₄ residues are connected to each other while sharing oxygen atoms are most preferable.

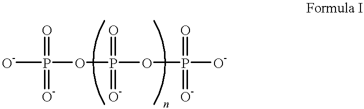

wherein, n is an integer from 3 to 798.

Also, polyphosphate salts in which hydrogen atoms of the hydroxy groups of the straight-chain polyphosphates are replaced by metals are useful. In this regard, sodium and potassium are found to be suitable for maintaining the therapeutic effect.

In accordance with the present invention, polyphosphate may be in a form of liquid, powder, gel or spray for application. As for the liquid application form, it may employs a harmless solvent selected from distilled water, physiological saline, buffers, etc.

However, the most effective results are obtained when an aqueous solution of polyphosphate is applied to wounds. In this case, the aqueous solution preferably has a concentration of 0.05-25%. For example, when the concentration of the aqueous polyphosphate solution is below 0.05%, the effect of wound healing and scar vanishment is insufficient. On the other hand, an aqueous polyphosphate solution with a concentration of greater than 25% cannot bring about a further improvement in the therapeutic effect, so it is economically unfavorable. In addition, such a highly concentrated solution is inconvenient to apply to wounds owing to its high viscosity.

The aqueous polyphosphate solution of the present invention is sufficiently applied to wounds at least three times a day, optionally in combination with known germicides and astringents to further enhance the effect of wound healing and scar declination. In this case, the germicides and astringents are dissolved in the aqueous polyphosphate solution and their kinds and amounts are determined through various experiments.

A better understanding of the present invention may be obtained in light of the following examples which are set forth to illustrate, but are not to be construed to limit the present invention.

### EXAMPLE 1

For experiments, adult SD rats were employed without discrimination of gender.

After being anesthetized, four rats were wounded to a length of 5-6 cm on their back with an operating knife. Only one suture was made in the central portion of each wound, after which a 4% aqueous solution of a mixture of polyphosphates with average chain lengths of 18 and 28 monomers in the proportions of 50:50 was sufficiently coated to the wound three times a day for three days.

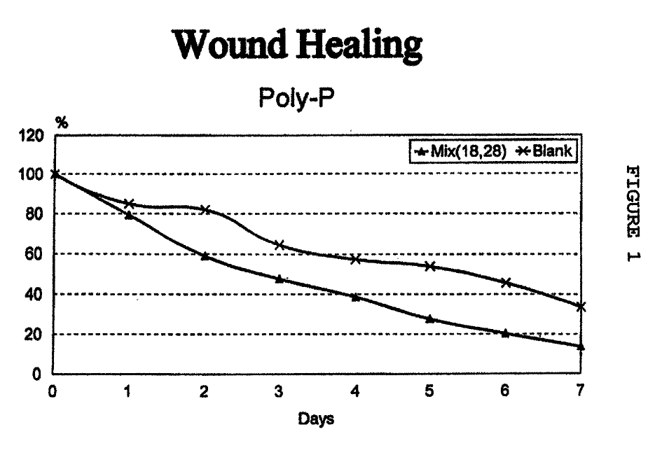

Next, 3, 6, 10, 14 and 15 days after the operation, average sizes of the wounds were measured in terms of surface area (width×length of the longest side in each wound) and from the measurements, % wound healing was calculated and depicted as shown in FIG. 1.

### EXAMPLE 2

As experimental animals, adult SD rats were employed without discrimination of gender.

After anesthesia, four rats were wounded to a length of 5-6 cm on their back with an operating knife. Only one suture was made in the central portion of each wound, after which a 10% aqueous solution of polyphosphate with an average chain length of 28 monomers was sufficiently coated to the wound three times a day for three days.

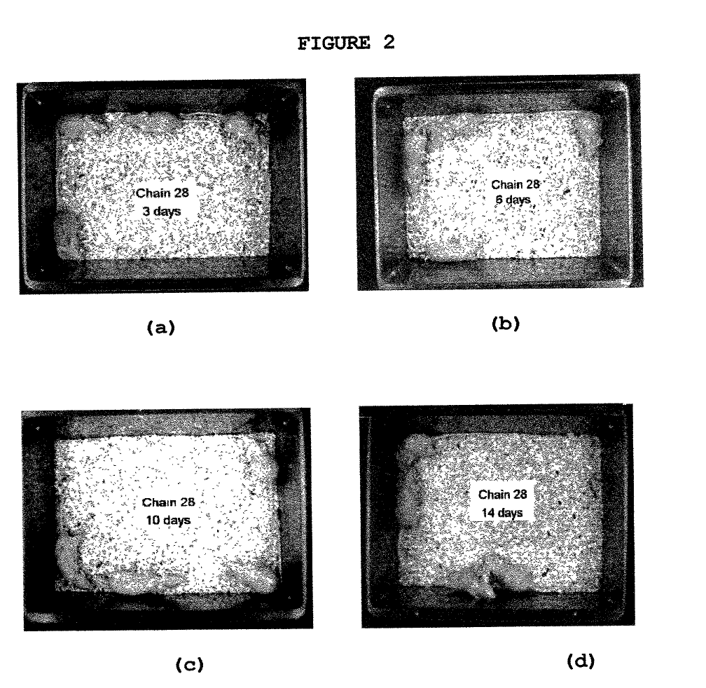

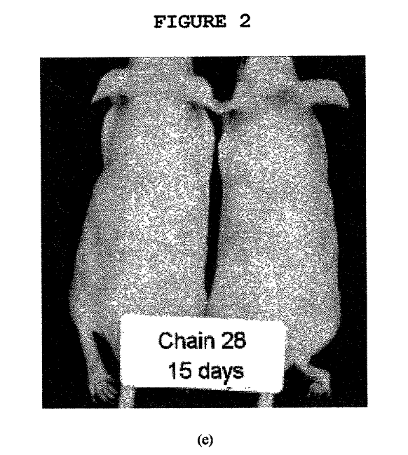

Next, 3, 6, 10, 14 and 15 days after the operation, average sizes of the wounds were measured in terms of surface area (width×length of the longest side in each wound) and the measurements are listed in Table 1, below. Also, photographs were taken of the wounds at the same times with the measurement to monitor their abatement and are shown in FIG. 2.

TABLE 1

| | 0 | 3^{rd} | 6^{th} | 10^{th} | 14^{th} | 15^{th} |
|---|---|---|---|---|---|---|
| | day | day | day | day | day | day |
| Wound Size | 500.0 | 475.3 | 352.6 | 139.2 | 102.4 | 77.4 |
| (mm²) | | | | | | |

### EXAMPLES 3 TO 6

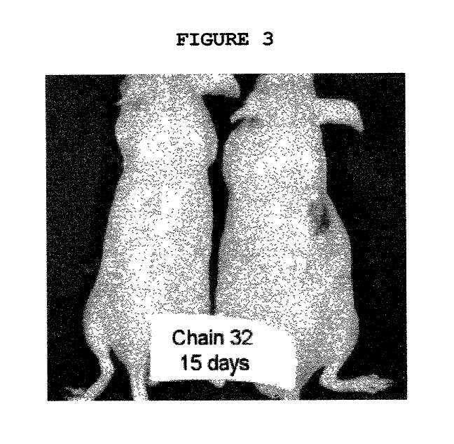

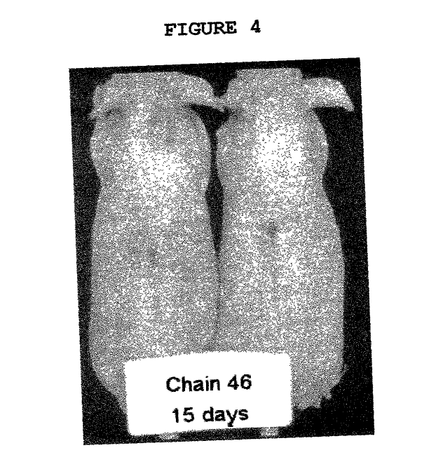

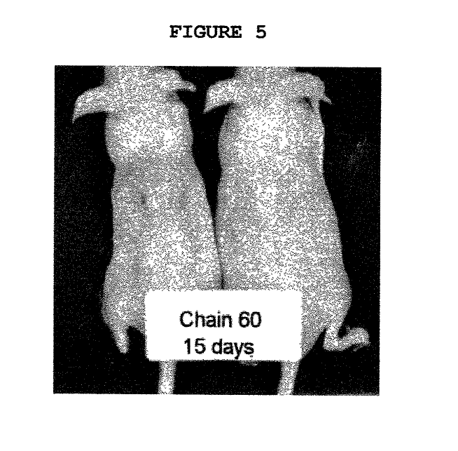

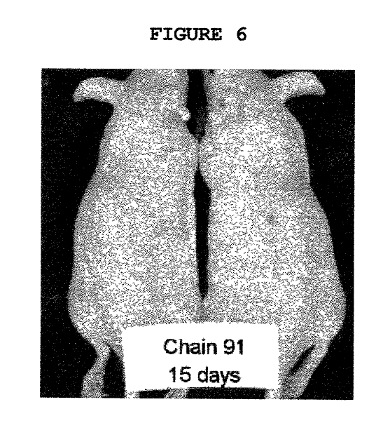

Wound treatment was conducted in manners similar to that of Example 1, except that aqueous solutions of polyphosphates with average chain lengths of 32, 46, 60 and 91 were used. 15 days after the operation, the wounds were measured for their sizes and the measurements are given in Table 2, below. Photographs of the wounds are shown in FIGS. 3 to 6.

TABLE 2

| | Example | Example | Example | Example |
|---|---|---|---|---|
| | 3 | 4 | 5 | 6 |
| Wound Size | 78.2 | 126.6 | 124.8 | 112.4 |
| (mm²) | | | | |

### COMPARATIVE EXAMPLE 1

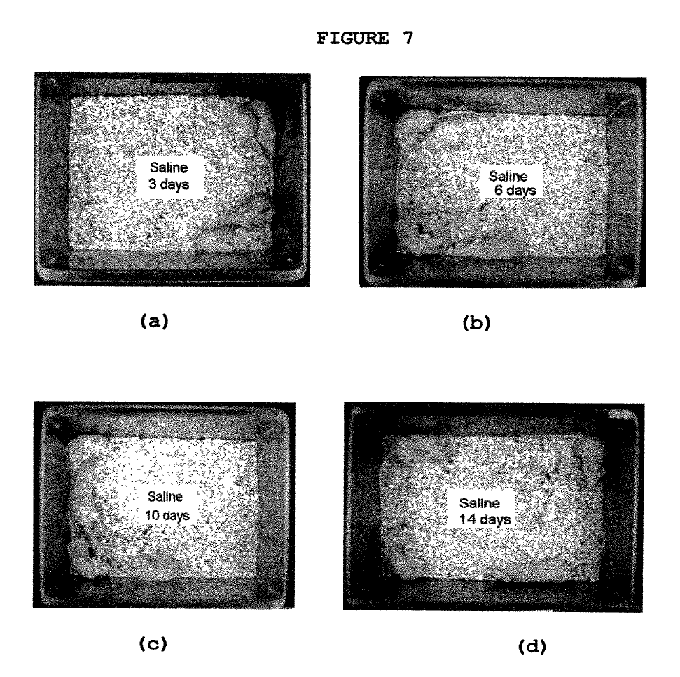

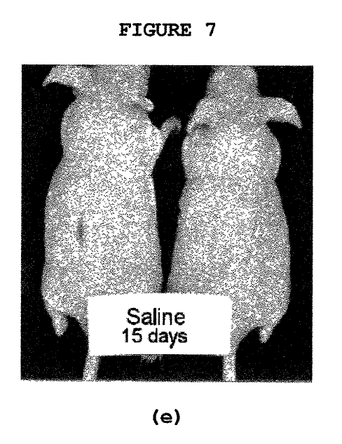

Wound treatment was conducted in a manner similar to that of Example 1, except that physiological saline was used instead of the aqueous polyphosphate solution. 3, 6, 10, 14 and 15 days after the operation, average sizes of the wounds were measured in terms of surface area (width×length of the longest side in each wound) and the measurements are listed in Table 3, below. Also, photographs were taken of the wounds at the same times as the measurement to monitor their abatement and are shown in FIG. 7.

TABLE 3

| | 0 | 3^{rd} | 6^{th} | 10^{th} | 14^{th} | 15^{th} |
|---|---|---|---|---|---|---|
| | day | day | day | day | day | day |
| Wound Size | 500.0 | 481.3 | 432.2 | 341.2 | 193.6 | 168.4 |
| (mm²) | | | | | | |

### COMPARATIVE EXAMPLE 2

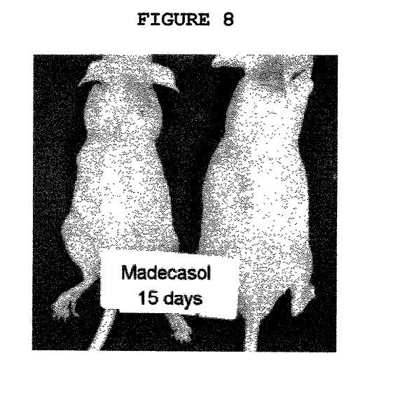

Wound treatment was conducted in a manner similar to that of Example 1, except that Madecassol Complex Ointment, a brand name of Dong Kook Pharm. Co. Ltd., was used instead of the aqueous polyphosphate solution. 15 days after the operation, the wound was measured to be 86.7 mm² in size and its photograph is shown in FIG. 8.

As apparent from the above examples, aqueous solutions of the polyphosphate represented by the chemical formula 1 can effectively promote the healing of wounds resulting from surgical operation or cutting without leaving scars, thereby encouraging patients to overcome loss of self-esteem. Compared with medicines for topical use, the medicinal aid of the present invention can heal wounds effectively and economically while reducing scarring.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A polyphosphate for use in promoting wound healing and scar abatement, comprising as therapeutically effective ingredient, represented by the following chemical formula 1: 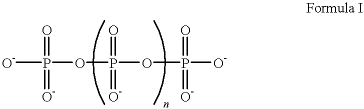
wherein n is an integer from 3 to 798.

2. The polyphosphate as set forth in claim 1, wherein said polyphosphate is a linear polyphosphate prepared through the dehydrocondensation of orthophosphates, a branched polyphosphate grafted with organic groups, or a cyclic polyphosphate.

3. The polyphosphate as set forth in claim 1, wherein said polyphosphate has metals substituted for hydrogen atoms of its hydroxy groups.

4. The polyphosphate as set forth in claim 3, wherein the metal is sodium or potassium.

5. The polyphosphate as set forth in claim 1, wherein the polyphosphate is in a form of an 0.05-25% aqueous solution.

6. A pharmaceutical composition comprising for use in promoting wound healing and scar abatement, comprising polyphosphate as therapeutically effective ingredient.
